(19) United States
(12) Patent Application Publication
(10) Pub. No.: US 2009/0247644 A1
(43) Pub. Date: Oct. 1, 2009

(54) MEMANTINE FORMULATIONS

(75) Inventors: Mahendra G. DEDHIYA, Pomona, NY (US); Ranajoy SARKAR, Piscataway, NJ (US)

Correspondence Address: Forest Laboratories, Inc.;Attn: Charles S. Ryan, 909 3rd Avenue, New York, NY, 10022, US

(73) Assignee: FOREST LABORATORIES HOLDINGS LIMITED

(21) Appl. No.: 12/412,447

(22) Filed: Mar. 27, 2009

Related U.S. Application Data
(60) Provisional application No. 61/040,171, filed on Mar. 28, 2008.

(51) Int. Cl. A61K 31/13

(52) U.S. Cl. 514/662

(57) ABSTRACT

The present invention relates to pharmaceutical compositions prepared from equant-shaped crystals of memantine, such as orally dissolving formulations, e.g., tablets (ODTs) and films (ODFs), and to methods of treating conditions, including childhood behavioral disorders (e.g., autism) and Alzheimer's disease by administering the same.

### FIELD OF THE INVENTION

The present invention relates to pharmaceutical compositions comprising equant-shaped crystals of memantine, such as orally dissolving formulations, e.g., tablets (ODTs) and films (ODFs), and to methods of treating conditions, including childhood behavioral disorders (e.g., autism) and Alzheimer's disease by administering the same.

### BACKGROUND OF THE INVENTION

It is known that the crystal habit and morphology, the external structure of a crystal, plays a significant role in packing, flowability, dissolution and sedimentation characteristics of solid pharmaceuticals. In general, there are several forms of crystal habits that crystals may exhibit. Some of the common known groups of crystal habits referenced in the United States Pharmacopoeia (USP) include planar (plate-like), acicular (needle-shaped) and equant (particles of roughly similar length, width and thickness, including both cubical and spherical particles). The morphology of the crystals may be determined by, e.g., optical microscopy (see USP Current Edition, Method 776). Crystals having the same polymorphic structure, i.e. the same unique arrangement of molecules inside the crystal, may still exhibit different crystal habits.

For a pharmaceutical substance to show a good tableting behavior, it is necessary that the substance (i) exhibit a low tendency to stick to the punches of the tableting press and (ii) have good flowability, in order to homogeneously fill the die. It is desirable, therefore, to crystallize a drug in a crystal habit which shows good flowability in order to allow for a smooth and reproducible tableting of the drug.

Although numerous publications exist dealing with the influence of solvent, temperature, stirring conditions, etc., on the crystallization behavior of drugs, it is difficult to identify the exact role of a single process variable on the crystal habit, because an alteration of one variable often leads to changes in the deposition and the dissolution rate of the molecules which both influence the crystal growth. Furthermore, there is currently no correlation known between, e.g., the type of solvent or the temperature and the resulting crystal habit of a substance.

Memantine (Namenda™) (1-amino-3,5-dimethyl adamantane), which is disclosed, e.g., in U.S. Pat. Nos. 4,122,193; 4,273,774; and 5,061,703, is a systemically-active uncompetitive NMDA receptor antagonist having low to moderate affinity for the receptor and strong voltage dependency and rapid blocking/unblocking kinetics. Memantine hydrochloride is currently available in the U.S. and in over 42 countries worldwide. It is approved for the treatment of moderate to severe Alzheimer's disease (AD) in the United States at a dose of up to 20 mg/day (5-10 mg BID). It has been hypothesized that memantine may not only be effective for the treatment of Alzheimer's disease (as well as Parkinson's and other neurological diseases), but may also be effective for the treatment of autism, attention deficit/hyperactivity disorder (ADHD) and other autistic spectrum disorders.

Various processes for the preparation of memantine are described, for example, in U.S. Pat. No. 4,122,193, U.S. Pat. No. 5,599,998, US 2005/0222271, US 2006/0173215, US2006/0217573, US2006/0258885, EP 392,095, CN 1,335,299, CZ 9601813, WO 2005/069742 and WO 2006/122238.

Crystals of memantine prepared in organic aprotic solvents using conventional synthetic processes have an acicular (needle-shaped) crystal habit. Such needle-shaped crystals exhibit a low flowability and a particle size varying from less than 10 μm to more than 800 μm. As a result, needle-shaped crystals have a tendency to form agglomerates and fine powders. The low flowability and large size variation of needle-shaped crystals of memantine create handling and metering difficulties, and are disadvantageous during the preparation of tablets and other dosage forms containing memantine because they can result in a higher variation in the average mass of the drug in the tablets and dosage forms. Also, it is well known that the acicular particles of pharmaceutical drugs are difficult to coat.

Therefore, it is desirable to provide memantine in a form which shows a high flowability to allow for a smooth and reproducible tableting of the drug. Applicants have found that equant-shaped crystals of memantine may be prepared, and that such crystals are highly suitable for tableting and other dosage forms and processes. It is also desirable to provide memantine in a form which can be coated with polymer, especially for modified release formulations including for the use in taste masking.

In addition, various formulation techniques have been used to provide sustained and immediate release of pharmaceutically active agents. In many such formulations, a drug-containing or drug-bearing particle is coated by one or more release retardant layers or is dispersed within a continuous matrix such as a polymeric matrix. The coating layer or the matrix comprises a relatively insoluble material or materials, and the release of the drug is controlled by means of the resistance or permeability of the coating layer or matrix against media entry into the formulation and subsequent diffusion of the drug. The release of the drug from such formulations is driven by diffusion, e.g., by the concentration gradient of the drug resulting from penetration of gastric fluid.

The use of orally dissolving formulations to administer pharmaceutical agents has also been disclosed. See, e.g., U.S. Pat. Nos. 3,784,390, 5,411,945, 5,980,882 and 6,001,392. Typically, the oral formulations contain a water-soluble polymer and other conventional excipients such as plasticizers and emulsifiers. However, the formulation composition will depend on the particular pharmaceutical agent and the desired formulation properties. For example, the formulation must be compatible with the pharmaceutical agent and also provide the necessary mechanical strength, taste-masking and dissolution properties.

Current dosing of memantine is twice a day using immediate release tablets. The tablet forms require the tablets to be coated to conceal the bitter taste of memantine. Moreover, the difficulties associated with multiple dosing of tablets result in decreased patient compliance, especially in autism patients and children. Orally dissolving formulations of memantine are beneficial for many reasons. Their characteristic advantages such as administration without liquid, anywhere, anytime lead to their suitability in situations where patients have difficulty swallowing, such as children, the elderly and, particularly, those with neurological disorders.

Thus, there is an existing and continual need for formulations containing memantine that provide reliable delivery and absorption of the active ingredient, while also providing a dosing regimen that is straightforward and increases patient compliance. Applicants have found that formulations, e.g., orally dissolving formulations, prepared from equant-shaped crystals of memantine show enhanced taste-masked properties, and are thus superior to similar formulations prepared from needle-shaped crystals of memantine.

### SUMMARY OF INVENTION

The present invention relates to pharmaceutical compositions comprising equant-shaped crystals of memantine, such as orally dissolving formulations, e.g., tablets (ODTs) and films (ODFs), and to methods of treating conditions, including childhood behavioral disorders (e.g., autism) and Alzheimer's disease by administering the same.

### BRIEF DESCRIPTION OF THE DRAWINGS

FIG. 1 shows a comparison of the particle size distribution of needle-shaped crystals of memantine hydrochloride and equant-shaped crystals of memantine hydrochloride.

### DETAILED DESCRIPTION OF THE INVENTION

In one aspect, the present invention relates to pharmaceutical compositions comprising equant-shaped crystals of memantine.

Memantine may preferably be used in the form of a pharmaceutically acceptable salt. Suitable salts of the compound include, but are not limited to, acid addition salts, such as those made with hydrochloric, methylsulfonic, hydrobromic, hydroiodic, perchloric, sulfuric, nitric, phosphoric, acetic, propionic, glycolic, lactic pyruvic, malonic, succinic, maleic, fumaric, maleic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. The term “salts” can also include addition salts of free acids or free bases. All of these salts (or other similar salts) may be prepared by conventional means. All such salts are acceptable provided that they are non-toxic and do not substantially interfere with the desired pharmacological activity.

As used herein “memantine” will be deemed to encompass both the free base and pharmaceutically acceptable salts thereof.

In certain embodiments, the present invention relates to pharmaceutical compositions comprising equant-shaped crystals of memantine hydrohalide, such as memantine hydrochloride or memantine hydrobromide, for example, memantine hydrochloride.

In additional embodiments, the equant-shaped crystals of memantine (e.g., memantine hydrochloride) have an aspect ratio of below about 5, below about 3, below about 2. In other embodiments, the equant-shaped crystals of memantine have an aspect ratio between about 1 and about 3, for example between about 1 and about 2.

In further embodiments, the mean particle diameter d₉₀ of the equant-shaped crystals of memantine is less than about 800 μm, such as less than about 500 μm, for example, less than about 400 μm. In further embodiments, the mean particle diameter d₉₀ of the equant-shaped crystals of memantine is from about 250 to about 500 μm, such as from about 300 to about 450 μm, for example, from about 350 to about 400 μm, e.g., about 375 μm.

In additional embodiments, the mean particle diameter d₅₀ of the equant-shaped crystals of memantine is from about 100 to about 500 μm, such as from about 200 to about 400 μm, for example, from about 200 to about 250 μm, e.g., about 205 μm.

In yet further embodiments, the mean particle diameter d₁₀ of the equant-shaped crystals of memantine is from about 1 to about 50 μm, such as from about 1 to about 20 μm, for example, from about 5 to about 15 μm, e.g., about 10 μm.

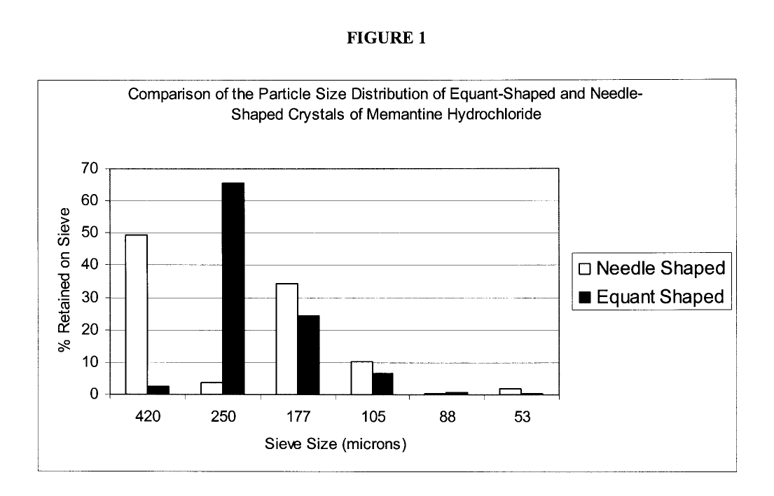

FIG. 1 shows a comparison of the particle size distribution of equant-shaped crystals of memantine hydrochloride and needle-shaped crystals of memantine hydrochloride.

In a further embodiment, the equant-shaped crystals of memantine have a specific surface area less than about 0.1 m²/g, such as less than about 0.05 m²/g. In further embodiments, the equant-shaped crystals of memantine have a specific surface area between about 0.01 and about 0.1 m²/g, such as between about 0.01 and about 0.05 m²/g, e.g., between about 0.02 and about 0.04 m²/g.

According to additional embodiments, the equant-shaped crystals of memantine exhibit an angle of repose of about 60 degrees or less, such as about 50 degrees or less, for example, about 45 degrees or less.

In a further embodiment, the equant-shaped crystals of memantine have a bulk density less than about 0.6 g/cm³, such as less than about 0.55 g/cm³. In other embodiments, the equant-shaped crystals of memantine have a bulk density from about 0.4 to about 0.6 g/cm³, such as from about 0.45 to about 0.55 g/cm³, for example, from about 0.48 to about 0.52 g/cm³. In yet further embodiments, the tap density of the equant-shaped crystals of memantine is from about 0.5 to about 0.7 g/cm³ such as from about 0.55 to about 0.65 g/cm³, for example, from about 0.57 to about 0.63 g/cm³.

The pharmaceutical compositions prepared using equant-shaped crystals of memantine may contain, for example, one or more pharmaceutically acceptable carriers. Suitable pharmaceutically acceptable carriers include diluents (such as sucrose, mannitol, lactose, starches) and excipients known in the art, including but not limited to suspending agents, solubilizers, buffering agents, binders, disintegrants, preservatives, colorants, flavorants, lubricants and the like

Numerous standard references are available that describe procedures for preparing various formulations. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration of the compositions of the present invention may be accomplished according to patient needs, for example, orally, nasally, parenterally (subcutaneously, intraveneously, intramuscularly, intrasternally and by infusion), by inhalation, rectally, vaginally, topically and by ocular administration.

Various solid oral dosage forms can be used, including such solid dosage forms as tablets, gelcaps, capsules, caplets, granules, films, lozenges and bulk crystalline powders.

In exemplary embodiments, the present invention relates to orally dissolving formulations, e.g., tablets (ODTs) and films (ODFs), comprising equant-shaped crystals of memantine, and to methods of treating conditions, including childhood behavioral disorders and Alzheimer's disease, by administering the orally dissolving formulations of the present invention.

According to some embodiments, the present invention provides orally dissolving formulations comprising at least one water soluble polymer and equant-shaped crystals of memantine.

In some embodiments, the orally dissolving formulations, e.g., tablets (ODTs) and films (ODFs), may be formulated so that the taste of the memantine is masked. In further embodiments, the formulations should meet the FDA guidelines for disintegration (See e.g., Food and Drug Administration, Center for Drug Evaluation and Research, Guidance for Industry Orally Disintegrating Tablets April 2007) and provide a desired bioavailability. For example, the orally dissolving formulations of the present invention may disintegrate within 30 seconds and be bioequivalent to existing tablet and liquid formulations of memantine, e.g., immediate release formulations.

In some embodiments, the orally dissolving formulations of the present invention may include about 1% to about 50% (by weight) memantine. In preferred embodiments, the orally dissolving formulations of the present invention may include about 5% to about 30% (by weight) memantine.

In some embodiments, the orally dissolving formulations of the present invention may include a water-soluble polymer, a combination of two or more water-soluble polymers or a combination of a water-soluble polymer and a water-insoluble or poorly-soluble polymer. Water soluble polymers that may be used in the orally dissolving formulations of the present invention include, but are not limited to, cellulose derivatives, synthetic polymers polyacrylates and natural gums. For example, the water soluble polymers used in the orally dissolving formulations of the present invention may include, but are not limited to, methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phtalate, cellulose acetate butyrate, amylose, dextran, casein, pullulan, gelatine, pectin, agar, carrageenan, xanthan gum, tragacanth, guar gum, acacia gum, arabic gum, polyethylene glycol, polyethylene oxide, polyvinyl pyrrolidone, polyvinyl alcohol, carboxyvinyl polymers, sodium alginate, polyacrylic acid, methylmethacrylate or mixtures thereof. In exemplary embodiments, the concentration of the water-soluble polymer in the formulation may be about 20% to about 90% (by weight), preferably between about 40% to about 80% (by weight).

In some embodiments, the orally dissolving formulations of the present invention may comprise an excipient. Suitable excipients include, but are not limited to, microcrystalline cellulose, colloidal silicon dioxide, talc, starch, sorbitol or combinations thereof. In some embodiments, the excipient may include talc as anti-adhering agent.

In some embodiments, the orally dissolving formulations of the present invention may comprise a plasticizer. Suitable plasticizers include, but are not limited to, polyethylene glycol, propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol or combinations thereof. In exemplary embodiments, the concentration of the plasticizer in the formulation may be about 0 to about 30 wt %, preferably about 0 to about 10 wt % and more preferably about 0 to about 4 wt %.

In some embodiments, the orally dissolving formulations of the present invention may comprise an emulsifying agent. As used herein, emulsifying agents include both solubilizers and wetting agents. Suitable emulsifying agents include, but are not limited to, polyvinyl alcohol, sorbitan esters, benzyl benzoate, glyceryl monostearate, polyoxyethylene alkyl ethers, polyoxyethylene stearates, poloxamer, polyoxyethylene castor oil derivatives (Cremophor), hydrogenated vegetable oils, bile salts, polysorbates, ethanol or combinations thereof.

In other embodiments, if present, the excipient is chosen to limit or avoid the formation of memantine adducts. As used herein, “adduct formation” refers to the formation of a compound with a particular formulation of a composition by a solid phase reaction. The general term “adduct” for a compound, also called an addition compound, results from the direct combination of two or more different compounds. For example, in the present invention, adduct formation may occur with formulations containing, for example, lactose (or other reducing sugars). Such adduct formation detracts from the efficacy of the product and increases the risks of other side effects.

In some embodiments, the orally dissolving formulations of the present invention may comprise a taste-masking agent. Generally, any natural or synthetic flavoring agent or sweetening agent known in the art may be used in the orally dissolving formulations of the present invention. For example, suitable taste-masking agents include, but are not limited to, essential oils, water soluble extracts, sugar, monosaccharides, oligosaccharides, aldose, ketose, dextrose, maltose, lactose, glucose, fructose, sucrose, mannitol xylitol, D-sorbitol, erythritol, pentitol, hexitol, malitol, acesulfame potassium, talin, glycyrrhizin, sucralose, aspartame, saccharin, sodium saccharin, sodium cyclamate, eugenyl formate aldehyde flavorings and combinations thereof.

Exemplary aldehyde flavorings that may be used include, but are not limited to acetaldehyde (apple); benzaldehyde (cherry, almond); cinnamic aldehyde (cinnamon); citral, i.e., alpha citral (lemon, lime); neral, i.e., beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e., trans-2 (berry fruits); tolyl aldehyde (cherry, almond); veratraldehyde (vanilla); 2,6-dimethyl-5-heptenal, i.e., melonal (melon); 2-6-dimethyloctanal (green fruit); and 2-dodecenal (citrus, mandarin). In some embodiments, the taste-masking agents may include combination of acesulfame potassium and flavors. One skilled in the art with the benefit of the present disclosure will appreciate that other and further ingredients may be included in the orally dissolving formulations of the present invention. For example, a matrix-forming polymer permeation enhancer, substance for imparting mucoadhesive properties, or other auxiliary substances disclosed, for example, in U.S. Patent Publication No. 2005/0163830.

In some embodiments, the orally dissolving formulations of the present invention may comprise memantine that has been coated. The coating may be used to mask the taste of the memantine or change the dissolution profile of the active ingredient. Any coating suitable for use in pharmaceutical formulations may be used. See, e.g., R. C. Rowe in Materials used in Pharmaceutical Formulation, Blackwell Scientific Publications, Oxford, 1, 36 (1984). Examples of suitable coating materials include polyethylene glycol, ethyl cellulose, methyl cellulose, hydroxypropyl methyl cellulose, acrylic resins, silicone elastomers, wax, fatty acids, polymethacrylate copolymers, Shellac, etc. In some embodiments, the coating may include between about 1% to about 75% of the formulation, preferably between about 10% to about 50% of the formulation.

In some embodiments, the orally dissolving formulations according to the present invention may include surfactants including, but not limited to, sodium docusate, polyoxyethylene ether, poloxamer, polysorbates (Tween), polyoxyethylene stearates, sodium lauryl sulfate, sorbitan esters and combinations thereof. If present, the surfactant may be included in the formulation from about 0.1% to about 10%, preferably between about 1% to about 5% (by weight). One skilled in the art, with the benefit of this disclosure, will understand that other components may be included to enhance one or more properties of the formulation. For example, the orally dissolving formulations according to the present invention may include disintegrating agents, antifoaming agents, antioxidants, buffering agents or coloring agents.

### Methods of Treatment

According to additional embodiments, the present invention provides methods of treatment comprising administering pharmaceutical compositions comprising equant-shaped crystals memantine to an individual in need thereof. For example, the compositions are suitable for the treatment of CNS disorders, including but not limited to the treatment of Alzheimer's disease, Parkinson's disease, AIDS dementia (U.S. Pat. Nos. 5,506,231, 5,061,703, and 5,614,560; see also Parsons et al., Neuropharmacology 1999 June; 38(6):735-67), neuropathic pain (U.S. Pat. No. 5,334,618), epilepsy, glaucoma, hepatic encephalopathy, multiple sclerosis, stroke, depression (U.S. Pat. No. 6,479,553), tardive dyskinesia, malaria, Borna virus, Hepatitis C (U.S. Pat. Nos. 6,034,134 and 6,071,966). Additional pathologies for treatment of which memantine is suitable are disclosed in U.S. Pat. Nos. 5,614,560 and 6,444,702.

Memantine may not only be effective for the treatment of Alzheimer's disease (as well as Parkinson's and other neurological diseases), but may also be effective for the treatment of autism, ADHD and other autistic spectrum disorders. See, for example, US 2006/0079582. The spectrum of childhood behavioral disorders include mental health problems such as anxiety disorders, Asperger's syndrome, ADHD, autistic spectrum disorders, autism, bipolar disorder, childhood disintegrative disorder, depression, disruptive behavior disorder, dyslexia, fragile X syndrome, learning disabilities, obsessive-compulsive disorder (OCD), oppositional defiant disorder, pervasive developmental disorder, reactive attachment disorder, Rett syndrome, separation anxiety disorder and Tourette's syndrome.

In some embodiments, the present invention provides methods for treating a disorder of the central nervous system by administering to a patient in need thereof pharmaceutical compositions comprising equant-shaped crystals of memantine. In exemplary embodiments, the present invention provides methods of treating childhood behavioral disorders, such as autistic spectrum disorders or combined type attention-deficit/hyperactivity disorder (ADHD). In other exemplary embodiments, the present invention provides methods of treating Alzheimer's disease. In additional exemplary embodiments, the present invention provides methods of treating autism.

The orally dissolving formulations of the present invention may release the memantine over a period of time that is determined by a number of different factors. These factors include the dimensions of the formulation, the concentration of the memantine, and how the memantine is dispersed throughout the formulation. For example, by varying the thickness and surface area of the formulations the rate of dissolution may be adjusted. A thick formulation, e.g. films, will dissolve more slowly than an otherwise similar thin formulation and may be desirable to administer high dosages of memantine. In some embodiments, water soluble inert filler may be used in the formulation to increase the solubility of the memantine. One skilled in the art with the benefit of this disclosure will realize that the extent of memantine uptake can be controlled by the dissolution rate of the formulation. In addition, the memantine may be released from the formulation and swallowed so it is also taken up in the GI tract.

In exemplary embodiments, the orally dissolving formulations of the present invention may dissolve after less than about 30 seconds. In yet other exemplary embodiments, the orally dissolving formulations may dissolve after less than about 20 seconds. In further embodiments, the dissolution rate of the active ingredient is more than about 80% (e.g., more than about 85%) within about the first 15 minutes following entry of the dosage form into a use environment.

In some embodiments, the memantine may be coated with a material to control the release of the memantine. Thus, the extent of memantine uptake can be controlled by the dissolution rate of the coated memantine. In other embodiments, the orally dissolving formulations of the present invention may include coated memantine or a mixture of coated and uncoated memantine. In exemplary embodiments, the coated memantine may be released from the formulation and swallowed so that uptake of the memantine occurs, partially or completely, in the GI tract.

### DEFINITIONS

The term “autism” refers to an individual demonstrating any one or all of the symptoms and characteristics associated with autism. Such individual may fit particular diagnostic criteria, such as Autistic Disorder, Asperger's Disorder, Atypical Autism or Pervasive Developmental Disorder, NOS (not otherwise specified), Rett's Disorder or Childhood Disintegrative Disorder, or the broader autism phenotype disorder or such individual may not fit a discrete diagnostic category at all. Due to the many presentations of the disease called autism, the present invention will use the term “autism” to refer to all of the above disorders.

As used herein, the terms “ODF,” “orally dissolving film,” and “orally disintegrating film” are used synonymously and mean that the film dissolves, melts, disintegrates, liquefies, etc. in the oral cavity such that substantially all of the memantine no longer remains in a formulation form.

The terms “ODT,” “orally dissolving tablet,” and “orally disintegrating tablet” are used synonymously and mean that the film dissolves, melts, disintegrates, liquefies, etc. in the oral cavity such that substantially all of the memantine no longer remains in a formulation form.

The “disintegration rate” is used herein to mean the amount of time that the film or tablet, dissolves, melts, disintegrates, liquefies, etc. in the environment of an oral cavity such that substantially all of the memantine no longer remains in a formulation form, e.g., in saliva at pH greater than 5.

The “dissolution rate” is used herein to mean the amount of time that it takes for the memantine, or pharmaceutically acceptable salt thereof, to become bioavailable.

A “therapeutically effective amount” means the amount of a compound that, when administered to a mammal for treating a state, disorder or condition is sufficient to effect a treatment (as defined below). The “therapeutically effective amount” will vary depending on the compound, the disease and its severity and the age, weight, physical condition and responsiveness of the mammal to be treated. According to the instant invention, in one embodiment, a therapeutically effective amount of memantine is an amount effective to treat CNS disorders, including Alzheimer's disease or Parkinson's disease. In another embodiment, a therapeutically effective amount is an amount effective to treat neuropathic pain, or other painful conditions such as visceral hypersensitivity. Other uses include, but are not limited to, the treatment of dementia, depression, and neuropathic pain. The effective amount of the drug for pharmacological action, and therefore the capsule strength, depends on the disease itself, e.g., in Alzheimer's disease, the patient is initially given a 5 mg dose and the dosage is progressively increased to 10 mg twice a day. Additional doses evaluated in clinical trials include 40 mg/day. In the present invention, e.g., in Alzheimer's disease treatment the patient may be initially given 2.5 mg and increased to 80 mg.

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans.

As used herein, the term “treat”, in all its verb forms, is used herein to mean to relieve or alleviate at least one symptom of a disorder in a subject, the disorder including for example, pain, Alzheimer's disease, vascular dementia, or Parkinson's disease. The term “treat” may mean to relieve or alleviate the intensity and/or duration of a manifestation of a disorder experienced by a subject in response to a given stimulus (e.g., pressure, tissue injury, cold temperature, etc.). For example, in relation to dementia, the term “treat” may mean to relieve or alleviate cognitive impairment (such as impairment of memory and/or orientation) or impairment of global functioning (activities of daily living, ADL) and/or slow down or reverse the progressive deterioration in ADL or cognition. Within the meaning of the present invention, the term “treat” also denote to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject. Within the meaning of the present invention, the dementia is associated with a CNS disorder, including without limitation neurodegenerative diseases such as Alzheimer's disease (AD), Down's Syndrome and cerebrovascular dementia (VaD). The term “treatment” means the act of “treating” as defined above.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value.

The term “entry into a use environment” means contact of a formulation of the invention with the saliva of a patient to whom it is administered, or with a fluid intended to simulate saliva.

A subject or patient in whom administration of the therapeutic compound is an effective therapeutic regimen for a disease or disorder is preferably a human, but can be any animal, including a laboratory animal in the context of a clinical trial or screening or activity experiment.

### Test Methods

### Aspect Ratio

The aspect ratio refers to the ratio of the maximum length of a crystal to its minimum width. At an aspect ratio of 1, a crystal has an isometric crystal habit. With the aspect ratio decreasing below 1, the crystal habit becomes more and more plate-like. In contrast, when the aspect ratio increases more and more above 1, the crystal approaches a needle-like crystal habit. In accordance with the present invention, cubic-shaped crystals of memantine hydrochloride have an aspect ratio of preferably below about 5, such as, below about 3 (3.0), e.g., below about 2 (2.0). The lower limit of the aspect ratio is not critical.

The aspect ratio is determined by means of scanning electron microscopy (SEM). The sample is spread onto a pure aluminum support and subsequently sputter-coated with a thin layer of Pd/Au (thickness in the nanometer range; sputter coater SCD 030 (Balzers)). Using a JSM 6400 Scanning Electron Microscope (JEOL, Japan) at an acceleration voltage of 20 keV and a working distance of 15 mm, SEM images are taken at 150-fold magnification. Electronic filtering and thresholding is used to improve the separation of particles in binary images from the background. For image analysis, ANALYSIS software version 5.0 (SIS, Muenster, Germany) is used. The software detects and assesses particles in the image automatically and classifies them according to the aspect ratio (range from 0.00 to 14; 14 classes in total). Also see, for example, USP Current Edition, Optical Microscopy, 776,

### Flowability

The flowability of the equant-shaped crystals of memantine was measured using a Jenike and Johanson Sifting Segregation tester (Jenike and Johanson, Tyngsboro, Mass.). The time taken for 200 g of material to fall through the circular orifice of the hopper was measured. Two different hoppers were used: a shallow angle cone (Hopper A, 35° from horizontal) and a steep angle cone (Hopper B, 75° from horizontal). Table 1 shows the results obtained for equant-shaped crystals of memantine hydrochloride and for needle-shaped crystals of memantine hydrochloride. As can be seen, the needle-shaped crystals do not flow through either cone type, whereas the equant-shaped crystals exhibit flow rates of about 38 g/s (Hopper A) and about 14 g/s (Hopper B). See, ASTM Standard D6941-03, and A. Alexander et al., Pharmaceutical Technology, Yearbook 2000, pp. 6-21.

TABLE 1

| | Equant-shaped | | Needle-shaped | |
|---|---|---|---|---|
| | crystals of | | crystals of | |
| | Memantine HCl | | Memantine HCl | |
| | Hopper A | Hopper B | Hopper A | Hopper B |
| | (Shallow | (Steep | (Shallow | (Steep |
| | Angle | Angle | Angle | Angle |
| | Cone) | Cone) | Cone) | Cone) |
| Flowability | 38.3 | 13.6 | Does not flow | Does not flow |
| (g/s) | | | | |

### Angle of Repose

For pharmaceutical powders, a lower angle of repose value indicates better flow characteristics. The angle of repose was measured using a Hosakawa Micron powder tester, Model PT-N (Hosokawa Micron Powder systems, Summit, N.J.) using the instructions provided by the vendor. Also see Pharmaceutical Dosage Forms, Tablets, Volume 1, Herbert A. Lieberman, Leon Lachman, Joseph B. Schwartz, 1989, 2nd Ed.

Bulk Density and Apparent Density after Tamping (Tap Density)

The bulk density and apparent density were measured using a Varian Tapped Density tester (Varian Inc.,) using the instructions provided by the vendor. Also see USP 29 (Tap-Bulk-Density).

### Particle Size Distribution

The particle size distribution (average d₉₀, d₅₀ and d₁₀ values) of the equant-shaped crystals of memantine was measured by Laser Diffraction technique using a Helos BF with Rodos instrument (Sympatec Inc, Lawrenceville, N.J.). The particle size distribution of the needle-shaped crystals of memantine was measured using a Mastersizer 2000 instrument (Malvern Instruments Limited, Worcestershire, UK).

The following examples are merely illustrative of the present invention and should not be construed as limiting the scope of the invention in any way as many variations and equivalents that are encompassed by the present invention will become apparent to those skilled in the art upon reading the present disclosure.

### EXAMPLES

### Example 1

### Synthesis of Equant-Shaped Crystals of Memantine

Equant-shaped crystals of memantine hydrochloride may be prepared, for example, by a procedure wherein a reactor is charged with needle-shaped crystals of memantine hydrochloride, 0.2 volumes of methanol, 2 volumes of ethyl acetate, and 1.4 volumes of distilled water. The resulting mixture is heated to 55-60° C. for 20-40 minutes (until a clear solution is obtained) and then filtered (using a cartridge filter to remove foreign particles) at this temperature into a reactor that is preheated to 55-60° C. The solution is cooled at a rate of 10° C. per hour. Precipitation is observed when the temperature is about 40° C. The suspension is further cooled to 20-25° C. at a rate of 10° C. per hour, then cooled to 0-5° C. over a period of 1 hour. The suspension is maintained at this temperature for at least 1 hour and then filtered. The filter cake is washed with 0.5-0.6 volumes of ethyl acetate. Drying under standard conditions yields equant-shaped crystals of memantine hydrochloride in 80-85 yield.

Table 2 compares the properties of equant-shaped crystals of memantine hydrochloride and needle-shaped crystals of memantine hydrochloride.

TABLE 2

| | Equant-shaped | Needle-shaped |
|---|---|---|
| | crystals of | crystals of |
| | Memantine HCl | Memantine HCl |
| Angle of Repose (°) | 43.1 | 66.2 |
| Bulk Density (g/cc) | 0.51 | 0.30 |
| Tap Density (g/cc) | 0.61 | 0.50 |
| Compressibility Index | 16 | 40 |
| PSD d₁₀ (μm) | 10 | 18.8 |
| PSD d₅₀ (μm) | 205 | 113.8 |
| PSD d₉₀ (μm) | 376 | 1229.4 |
| Aspect Ratio | 1.6 | 11.4 |

The results in Table 2 demonstrate that equant-shaped crystals of memantine hydrochloride exhibit good flowability compared to the poorly flowable needle-like crystals of memantine hydrochloride. The equant-shaped crystals have lower angle of repose compared to the needle-like crystals. The bulk and tap densities are greater for the equant-shaped crystals and the Compressibility Index is much lower for the equant-shaped crystals. Further, the equant-shaped crystals show a significantly narrower particle size distribution in comparison to the needle-like crystals. The superior flowability and physical characteristics of the equant-shaped crystals make them especially suited for the solid dosage forms, such as capsules and tablets.

### Example 2

In an attempt to mask the bitter taste of memantine, equant-shaped crystals of memantine hydrochloride and needle-shaped crystals of memantine hydrochloride were independently dry coated with taste masking and flavoring agents e.g. Mimic Nat Powder, Neohesperidine, MR-77 Masking Fl, MW-94 Masking NFF, MQ-82 Prosweet Fl, Lemon Juice powder, Lemon Crystarome the components being blended for 5 minutes. See Table 3.

TABLE 3

| | Needle-like | Equant-shaped |
|---|---|---|
| | crystals of | crystals of |
| Ingredient | Memantine HCl | Memantine HCl |
| Active Ingredient (g) | 8.0 | 8.0 |
| Flavoring & Taste | 1.3 | 1.3 |
| Masking Agents (g) | | |
| Total (g) | 9.3 | 9.3 |

The coated particles were tasted (n=2), without the individuals receiving a dose of the drug, to determine the effectiveness of the taste-masking. The bitter taste of the needle-like crystals was perceived immediately (in less than 5 seconds). The bitter taste of equant-shaped crystals could not be perceived for 10 seconds upon tasting.

### Example 3

In a further attempt to mask the bitter taste of memantine, equant-shaped crystals of memantine hydrochloride and needle-like crystals of memantine hydrochloride were independently directly coated with methyl methacrylate-butyl methacrylate-dimethylaminoethyl methacrylate copolymer, Eudragit E (Degussa, Rohm Pharma Polymers, N.J.) as the taste-masking polymer. Eudragit E is a cationic polymer and is soluble below a pH of 5 and swellable and permeable above pH of 5. Therefore, this polymer dissolves readily in stomach (pH 1-3) but resists dissolution at saliva (pH greater than 5).

The drug particles (400 g) were loaded into the bowl of a Glatt Fluid Bed Coater (GPGC 3.1, Glatt Air Technique, Ramsey, N.J.). Eudragit dispersion was prepared according to manufacturer's instructions (Degussa, Piscataway, N.J.). Memantine drug substance was coated with the following conditions: Inlet Air Temperature 40 to 50° C.; Product Temperature 27 to 32° C.; Atomization pressure 1 to 2 Bars; Spray rate between 6-12 grams per minute; Target weight gain was 50% w/w. The resulting drug product had up to a 50% weight gain of the taste-masking Eudragit polymer. The drug product composition is shown in Table 4.

TABLE 4
Composition of Memantine HCl Particles Coated with 50% w/w
Eudragit Polymer.

| Ingredient | Weight (g) |
|---|---|
| Memantine HCl | 400 |
| Methyl methacrylate-butyl | 125 |
| methacrylate-dimethylaminoethyl | |
| methacrylate copolymer (Eudragit E) | |
| Sodium Lauryl Sulfate | 12.5 |
| Stearic Acid | 18.75 |
| Mg Stearate | 43.75 |
| Total | 600 |

The coated particles were tasted (n=2), without the individuals receiving a dose of the drug, to determine the effectiveness of the taste-masking. The bitter taste of the needle-like crystals was readily perceived (in less than 5 seconds), showing that the method did not mask the bitter taste of the needle-shaped crystals satisfactorily. Although this method is widely used, it is not effective for direct coating of the needle-shaped particles of memantine because the end portions of the needles are especially difficult to coat and causes the drug to leach into the mouth. In addition, the coating process is difficult to control with needle-shaped crystals because they tend to fracture easily during processing, leading to creation of uncoated surfaces.

On the other hand, the bitter taste of equant-shaped crystals could not be perceived for 30 seconds (at coating levels of 50%). This shows that the equant-shaped crystals of memantine were effectively taste masked at a weight gain of 50% w/w.

### Example 4

An orally dissolving film comprising equant-shaped memantine was prepared by dissolving polyethylene oxide in water followed by the addition of plasticizer (polyethylene glycol), The taste-masked equant-shaped memantine hydrochloride particles, as prepared in Example 3, were then added and mixed for about 10 minutes before casting the film on a Teflon surface using a BYK-Gardner film casting knife (Columbia, Md.). The film was dried in oven first at 80° C. for 15 minutes and then at 50° C. until dried. The films were then cut to size so that each piece contained a dose ranging from 2.5 mg to 80 mg. Table 5 shows the composition of the orally dissolving films of equant-shaped memantine.

TABLE 5
Orally Dissolving Films of Equant-Shaped Memantine HCl
(Without Sweeteners)

| Ingredient | Function | 3 mg | 6 mg | 12 mg | 24 mg |
|---|---|---|---|---|---|
| Equant-shaped Memantine | Active | 4.5 | 9.0 | 18.0 | 36.0 |
| Taste Masked Granules | | | | | |
| Polyethylene Oxide | Film | 16.9 | 33.8 | 67.6 | 135.2 |
| (Mol wt = 200,000 and | former | | | | |
| 100,000) | | | | | |
| Polyethylene Glycol 400 | Plasticizer | 1.1 | 2.2 | 4.4 | 8.8 |
| Water (evaporated during | Solvent | QS | QS | QS | QS |
| processing) | | | | | |
| Total | | 22.5 | 45 | 90 | 180 |

The orally dissolving films were tasted (n=2), without the individuals receiving a dose of the drug, to determine the effectiveness of taste masking. The bitterness of the drug was not noticeable upon tasting showing that orally dissolving films of equant-shaped memantine were effectively taste-masked using the described approach.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

The disclosures of all patents, patent applications and publications cited throughout this application are incorporated herein by reference in their entireties.

1. A pharmaceutical composition comprising equant-shaped crystals of memantine and a pharmaceutically acceptable carrier.

2. The pharmaceutical composition according to claim 1, comprising memantine hydrochloride.

3. The pharmaceutical composition according to claim 1, wherein the equant-shaped crystals of memantine have an aspect ratio below about 5.

4. The pharmaceutical composition according to claim 1, wherein the equant-shaped crystals of memantine have an aspect ratio between about 1 and about 2.

5. The pharmaceutical composition according to claim 1, wherein the equant-shaped crystals of memantine have a specific surface area of less than about 0.1 m²/g.

6. The pharmaceutical composition according to claim 1, wherein the equant-shaped crystals of memantine have an angle of repose of about 60 degrees or less.

7. The pharmaceutical composition according to claim 1, wherein the equant-shaped crystals of memantine have an angle of repose of about 50 degrees or less.

8. The pharmaceutical composition according to claim 1, wherein the equant-shaped crystals of memantine have an angle of repose of about 45 degrees or less.

9. The pharmaceutical composition according to claim 1, wherein the average particle size d₉₀ of the equant-shaped crystals of memantine is less than about 800 μm.

10. The pharmaceutical composition according to claim 1, wherein the bulk density of the equant-shaped crystals of memantine is from about 0.45 to about 0.55 g/cc.

11. The pharmaceutical composition according to claim 1, wherein the tap density of the equant-shaped crystals of memantine is from about 0.55 to about 0.65 g/cc.

12. An orally dissolving pharmaceutical composition comprising the pharmaceutical composition of claim 1, and at least one water soluble polymer.

13. The orally dissolving pharmaceutical composition of claim 12, wherein the water soluble polymer is selected from the group consisting of methyl cellulose, hydroxypropyl cellulose, hydroxypropylmethyl cellulose, ethyl cellulose, hydroxyethyl cellulose, hydroxypropyl cellulose, carboxymethyl cellulose, cellulose acetate phtalate, cellulose acetate butyrate, amylose, dextran, casein, pullulan, gelatine, pectin, agar, carrageenan, xanthan gum, tragacanth, guar gum, acacia gum, arabic gum, polyethylene glycol, polyethylene oxide, polyvinyl pyrrolidone, polyvinyl alcohol, carboxyvinyl polymers, sodium alginate, polyacrylic acid, methylmethacrylate and mixtures thereof.

14. The orally dissolving pharmaceutical composition of claim 12, further comprising a taste masking agent, a flavoring agent, a softener, a diluent, a stabilizer, a dye, a colorant, a disintegrant. an excipient, or combinations thereof.

15. The orally dissolving pharmaceutical composition of claim 12, wherein the formulation is a film.

16. The orally dissolving pharmaceutical composition of claim 12, wherein the formulation is a tablet.

17. The orally dissolving pharmaceutical composition of claim 12, wherein the memantine is taste-masked.

18. The orally dissolving pharmaceutical composition of claim 12, wherein the dissolution rate of the active ingredient is more than about 80% within about the first 15 minutes following entry of the dosage form into a use environment.

19. The orally dissolving pharmaceutical composition of claim 12, wherein the dissolution rate of the active ingredient is more than about 85% within about the first 15 minutes following entry of the dosage form into a use environment.

20. The orally dissolving pharmaceutical composition of claim 12, wherein the disintegration rate of formulation is less than 30 seconds following entry of the dosage form into a use environment.

21. The orally dissolving pharmaceutical composition of claim 12, wherein the disintegration rate of formulation is less than 15 seconds following entry of the dosage form into a use environment.

22. A method for treating a disorder of the central nervous system, comprising administering to a patient in need thereof a pharmaceutical composition of claim 1.

23. The method of claim 22, wherein the disorder of the central nervous system is Alzheimer's disease.

24. A method for treating a childhood behavioral disorder, comprising administering to a patient in need thereof a pharmaceutical composition of claim 1.

25. The method of claim 24, wherein the childhood behavioral disorder is autism.

26. A method for treating a disorder of the central nervous system, comprising administering to a patient in need thereof the orally dissolving formulation of any claim 1.

27. The method of claim 26, wherein the disorder of the central nervous system is Alzheimer's disease.

28. A method for treating a childhood behavioral disorder, comprising administering to a patient in need thereof the orally dissolving formulation of claim 12.

29. The method of claim 28, wherein the childhood behavioral disorder is autism.
